(19) United States
(12) Patent Application Publication
(10) Pub. No.: US 2025/0285712 A1
(43) Pub. Date: Sep. 11, 2025

(54) METHOD AND APPARATUS FOR PREDICTING PROTEIN-LIGAND BINDING FREE ENERGY

(71) Applicants: INCEREBRO, INC., Seoul (KR); KOREA UNIVERSITY RESEARCH AND BUSINESS FOUNDATION, SEJONG CAMPUS, Sejong-si (KR)

(72) Inventors: Eun Sung CHO, Seoul (KR); Farzad MOLANI, Sejong-si (KR)

(73) Assignees: INCEREBRO, INC., Seoul (KR); KOREA UNIVERSITY RESEARCH AND BUSINESS FOUNDATION, SEJONG CAMPUS, Sejong-si (KR)

(21) Appl. No.: 18/598,226

(22) Filed: Mar. 7, 2024

(51) Int. Cl. G16C 10/00; G06F 30/20; G16B 15/30

(52) U.S. Cl. CPC G16C 10/00; G06F 30/20; G16B 15/30

(57) ABSTRACT

Provided are a method and apparatus for predicting protein-ligand binding free energy. The method includes generating a highest-probability pose for a selected protein-ligand set using a quantum mechanics-based Mining Minima algorithm, replacing a charge value of the pose with atomic electrostatic potential charge (Q_{ESP}) using a quantum mechanical calculation method predicting the atomic charges of protein residues and ligands existing in a quantum mechanical domain, and predicting the protein-ligand binding free energy using the charge value-replaced pose.

### BACKGROUND

### 1. Field

The present disclosure relates to a method and apparatus for predicting protein-ligand binding free energy.

### 2. Description of the Related Art

The use of protein-ligand binding affinity calculations has been increasing since the first study about 30 years ago (Merz Jr, K. M. and P. A. Kollman, Journal of the American Chemical Society, 1989. 111 15): p. 5649-5658). In particular, fast and accurate prediction of non-covalent receptor-ligand binding free energy (FE) is important not only in academic research, but also in drug development (Xie, B., T. H. Nguyen, and D. D. Minh, Journal of chemical theory computation, 2017. 13(6): p. 2930-2944). Methods in this technical field include Free Energy Perturbation (FEP), Linear Response (LR), Thermodynamic Integration (TI), Statistical mechanics-based methods, and MM-PB/GB-SA (molecular mechanics energies combined with Poisson-Boltzmann or generalized Born and surface area continuum solvation).

Recently, researchers tested the accuracy of the FEP protocols for calculating relative binding free energy and absolute binding free energy (ABFE). They obtained average Pearson correlations (R-values) of 0.61, 0.72, and 0.60 for the receptors under study in their particular cases. However, the usefulness of these FEP protocols is limited by the computational cost and their accuracy depends on the quality of the force field (FF) used.

The background technology described above is technical information that the inventor possessed for deriving the invention or acquired in the process of deriving the invention, and cannot be necessarily said to be known to the general public prior to the filing of the invention.

### SUMMARY

Provided is a method of predicting protein-ligand binding free energy. Provided is a computer-readable recording medium on which a program for executing the method on a computer is recorded.

The objective of the present disclosure is not limited to the objectives mentioned above, and other objectives and advantages of the present disclosure that are not mentioned can be understood through the following description and will be understood more clearly through the embodiments of the present disclosure. In addition, it will be appreciated that the objectives and advantages of the present disclosure can be realized by the means described in the claims, and combinations thereof.

Additional aspects will be set forth in part in the description which follows and, in part, will be apparent from the description, or may be learned by practice of the presented embodiments of the disclosure.

According to an aspect of an embodiment, a method of predicting protein-ligand binding free energy includes generating the highest-probability pose for a selected protein-ligand set using a quantum mechanics-based Mining Minima algorithm; replacing a charge value of the pose with atomic electrostatic potential charge (Q_{ESP}) using a quantum mechanical calculation method predicting the atomic charges of protein residues and ligands existing in a quantum mechanical domain; and predicting the protein-ligand binding free energy using the charge value-replaced pose.

According to an aspect of an embodiment, an apparatus for predicting protein-ligand binding free energy includes at least one memory; and at least one processor, wherein the at least one processor is configured to generate the highest-probability pose for a selected protein-ligand set using a quantum mechanics-based Mining Minima algorithm, replace a charge value of the pose with atomic electrostatic potential charge (Q_{ESP}) using a quantum mechanical calculation method predicting the atomic charges of protein residues and ligands existing in a quantum mechanical domain, and predict the protein-ligand binding free energy using the charge value-replaced pose.

According to an aspect of an embodiment, provided is a computer-readable recording medium on which a program for executing the method according to the first aspect on a computer is recorded.

According to an aspect of an embodiment, provided are other methods, systems, and computer-readable recording mediums storing a computer program for executing the method for implementing the present disclosure may be further provided.

Other aspects, features and advantages in addition to those described above will become apparent from the following drawings, claims and detailed description of the present disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram illustrating an example of a system for predicting protein-ligand binding free energy according to an embodiment;

FIG. 2 is a configuration diagram illustrating an example of a user terminal according to an embodiment;

FIG. 3 is a flowchart illustrating an example of a method of predicting protein-ligand binding free energy according to an embodiment;

FIG. 4 is a diagram illustrating the relative binding free energy predicted by a processor according to an embodiment;

FIG. 5 is a diagram for comparing relative binding free energies predicted by the processor according to an embodiment;

FIG. 6 is a diagram illustrating the experimental binding energy and the binding energy predicted by the MM-VM2 method;

FIG. 7 is a diagram illustrating the experimental binding energy and the binding energy predicted by a processor according to an embodiment using the Qcharge-VM2 method;

FIG. 8 is a diagram illustrating the experimental binding energy and an absolute error offset according to an embodiment; and

FIG. 9 is a diagram illustrating another example of a method of predicting protein-ligand binding free energy according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Various embodiments of the present disclosure are described below in conjunction with the accompanying drawings. Since various embodiments of the present disclosure can make various changes and have various embodiments, specific embodiments are illustrated in the drawings and related detailed descriptions are described. However, it should be understood that this is not intended to limit the various embodiments of the present disclosure to the specific embodiments, and the embodiments include all changes and/or equivalents or substitutes included in the spirit and technical scope of the various embodiments of the present disclosure. In connection with the description of the drawings, similar reference numerals have been used for similar components.

As used in various embodiments of the present disclosure, terms such as “include,” or “including” indicate the existence of the corresponding function, operation, or component, and do not limit one or more additional functions, operations, or components. It will be further understood that the terms “include,” “have,” etc., when used in various embodiments of the present disclosure, specify the presence of stated features, integers, steps, operations, elements, components, or combinations thereof, but do not preclude the presence or addition of one or more other features, integers, steps, operations, elements, components, or combinations thereof.

As used in various embodiments of the present disclosure, expressions such as “or” include any or all combinations of words listed together. For example, “A or B” may include A, B, or both A and B.

As used in various embodiments of the present disclosure, expressions such as “first,” “second,” “primary,” or “secondary” may refer to various components of the various embodiments, but are not intended to limit such components. For example, the above expressions do not limit the order and/or importance of the corresponding components. The above expressions can be used to distinguish one component from another. For example, a first user device and a second user device are both user devices and represent different user devices. For example, a first component may be referred to as a second component, and similarly, a second component may be referred to as a first component without departing from the scope of various embodiments of the present disclosure.

It will be understood that when an element is referred to as being “coupled” or “connected” to another element, it can be directly coupled or connected to the other element or intervening other elements may be present therebetween. In contrast, it should be understood that when an element is referred to as being “directly coupled” or “directly connected” to another element, there are no intervening other elements present.

As used in embodiments of the present disclosure, terms such as “module,” “unit,” “part,” etc. are terms to refer to components that perform at least one function or operation, and these components may be implemented in hardware, software, or a combination of hardware and software. In addition, a plurality of “modules,” “units,” “parts,” or the like may be integrated into at least one module or chip and implemented as at least one processor, except in cases where each needs to be implemented in separate and specific hardware.

Terms used in various embodiments of the present disclosure are merely used to describe specific embodiments and are not intended to limit the various embodiments of the present disclosure. Singular expressions include plural expressions unless the context clearly dictates otherwise.

Some embodiments of the present disclosure may be represented by functional block configurations and various processing steps. Some or all of these functional blocks may be implemented in various numbers of hardware and/or software configurations that perform specific functions. For example, the functional blocks of the present disclosure may be implemented by one or more microprocessors, or may be implemented by circuit configurations for certain functions. Additionally, for example, the functional blocks of the present disclosure may be implemented in various programming or scripting languages. Functional blocks may be implemented as an algorithm running on one or more processors. Additionally, the present disclosure may employ conventional technologies for electronic environment setup, signal processing, and/or data processing. Terms such as “mechanism,” “element,” “means,” and “configuration” may be used broadly and are not limited to mechanical and physical configurations.

Additionally, connection lines or connection members between components illustrated in the drawings merely exemplify functional connections and/or physical or circuit connections. In an actual device, connections between components may be represented by various replaceable or additional functional connections, physical connections, or circuit connections.

Unless otherwise defined, all terms used herein, including technical or scientific terms, have the same meaning as commonly understood by a person of ordinary skill in the technical field to which the various embodiments of the present disclosure pertain.

Terms such as those defined in commonly used dictionaries are to be construed to have meanings consistent with their meaning in the context of the relevant art, and are not to be construed in an idealized or overly formal sense unless expressly defined in the various embodiments of this disclosure.

Hereinafter, various embodiments of the present disclosure will be described in detail with reference to the accompanying drawings.

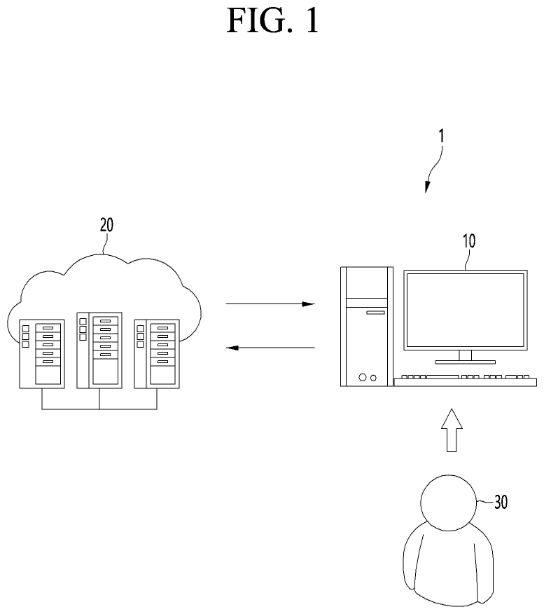

FIG. 1 is a diagram illustrating an example of a system 1 for predicting protein-ligand binding free energy according to an embodiment.

Referring to FIG. 1, the system 1 includes a user terminal 10 and a server 20. For example, the user terminal 10 and the server 20 may be connected through wired or wireless communication to transmit and receive data (for example, data corresponding to a protein-ligand set, etc.).

For convenience of explanation, FIG. 1 shows that the system 1 includes a user terminal 10 and a server 20, but the system 1 is not limited thereto. For example, the system 1 may include other external devices (not shown), and the operations of the user terminal 10 and the server 20, which will be described below, may be performed using a single device (e.g., the user terminal 10, or the server 20).

The user terminal 10 may be a computing device that includes a display device and a device (e.g., a keyboard, a mouse, etc.) receiving user input, as well as memory, and a processor. For example, the user terminal 10 may include, but is not limited to, a notebook PC, a desktop PC, a laptop, a tablet computer, a smart phone, etc.

The server 20 may be a device that communicates with external devices (not shown), including the user terminal 10. For example, the server 20 may be a device that stores various data, including protein-ligand sets, and in some cases, may be a device with its own computing capabilities. For example, the server 20 may be a cloud server, but is not limited thereto.

The user terminal 10 is configured to generate the highest-probability pose for a selected protein-ligand set using a quantum mechanics-based Mining Minima algorithm, replace a charge value of the pose with atomic electrostatic potential charge (Q_{ESP}) using a quantum mechanical calculation method predicting the atomic charges of protein residues and ligands existing in a quantum mechanical domain, and predict the protein-ligand binding free energy using the charge value-replaced pose.

According to an embodiment, “docking” generally refers to a computational simulation of the binding of a candidate ligand to a receptor (e.g., a protein). In the field of molecular modeling, docking is used as a way to predict the preferred orientation of a first molecule with respect to a second molecule when two molecules bind to each other to form a stable complex.

According to an embodiment, a “pose” refers to a candidate binding mode generated by docking.

According to an embodiment, “scoring” refers to a process of evaluating the ranking of a specific pose created between two molecules after docking in the field of computational chemistry and molecular modeling.

According to an embodiment, a “quantum mechanical domain” may be defined as including protein residues and ligands at the binding site between the protein and ligand.

The system 1 according to an embodiment predicts the protein-ligand binding free energy using the pose with the charge value replaced by Q_{ESP}. In some embodiments, the user terminal 10 is operated to generate the highest-probability pose for a selected protein-ligand set using a quantum mechanics-based Mining Minima algorithm, replace a charge value of the pose with atomic electrostatic potential charge (Q_{ESP}) using a quantum mechanical calculation method predicting the atomic charges of protein residues and ligands existing in a quantum mechanical domain, and predict the protein-ligand binding free energy using the charge value-replaced pose. The user 30 may determine information about the protein-ligand binding free energy using the highest-probability pose of which charge value is replaced.

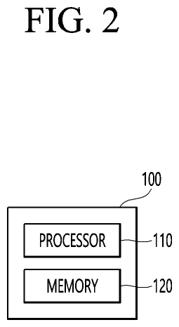

Hereinafter, with reference to FIGS. 2 to 8, a description will be made of examples of operation of the user terminal 10 to generate the highest-probability pose for a selected protein-ligand set using a quantum mechanics-based Mining Minima algorithm, replace a charge value of the pose with atomic electrostatic potential charge (Q_{ESP}) using a quantum mechanical calculation method predicting the atomic charges of protein residues and ligands existing in a quantum mechanical domain, and predict the protein-ligand binding free energy using the charge value-replaced pose. Meanwhile, as described above with reference to FIG. 1, operations to be described later with reference to FIGS. 2 to 8 may be performed in the server 20.

FIG. 2 is a configuration diagram illustrating an example of a user terminal according to an embodiment.

Referring to FIG. 2, the user terminal 100 includes a processor 110 and a memory 120. For convenience of explanation, only components related to the present disclosure are illustrated in FIG. 2. In addition to the components illustrated in FIG. 2, other general-purpose components may be further included in the user terminal 100. As an example, the user terminal 100 may include an input/output interface (not shown) and/or a communication module (not shown). Additionally, it is obvious to those skilled in the art that the processor 110 and memory 120 illustrated in FIG. 2 may be implemented as independent devices.

The processor 110 may process computer program instructions by performing basic arithmetic, logic, and input/output operations. Here, the instructions may be provided from the memory 120 or an external device (e.g., the server 20, etc.). Additionally, the processor 110 may generally control the operations of other components included in the user terminal 100.

In some embodiments, the processor 110 may use the pose to predict protein-ligand binding free energy. In some embodiments, the processor 110 may generate the highest-probability pose for a selected protein-ligand set using a quantum mechanics-based Mining Minima algorithm. In some embodiments, the processor 110 may replace a charge value of the pose with atomic electrostatic potential charge (Q_{ESP}) using a quantum mechanical calculation method predicting the atomic charges of protein residues and ligands existing in a quantum mechanical domain. Then, the processor 110 may predict the protein-ligand binding free energy using the charge value-replaced pose.

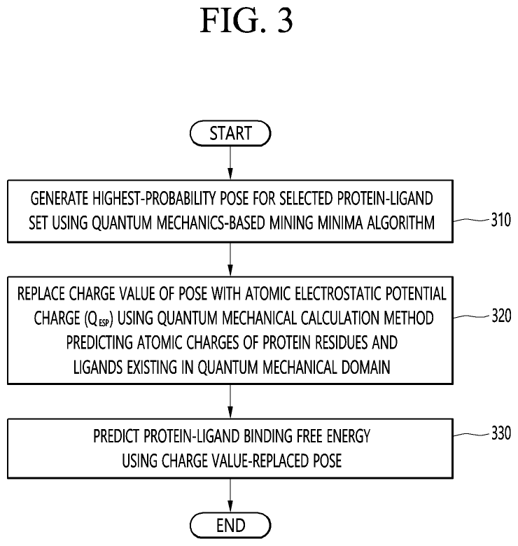

Specific examples of operation of the processor 110 according to an embodiment will be described with reference to FIGS. 3 to 8.

The processor 110 may be implemented as an array of multiple logic gates, or may be implemented as a combination of a general-purpose microprocessor and a memory storing a program that may be executable on the microprocessor. For example, the processor 110 may include a general purpose processor, a central processing unit (CPU), a microprocessor, a digital signal processor (DSP), a controller, a microcontroller, a state machine, etc. In some circumstances, the processor 110 may include an application specific integrated circuit (ASIC), a programmable logic device (PLD), a field programmable gate array (FPGA), etc. For example, the processor 110 may refer to a combination of processing devices, such as a combination of a digital signal processor (DSP) and a microprocessor, a combination of a plurality of microprocessors, a combination of one or more microprocessors combined with a digital signal processor (DSP) core, or a combination of any other such configurations.

The memory 120 may include any non-transitory computer-readable recording medium. As an example, the memory 120 may include a permanent mass storage device such as random access memory (RAM), read only memory (ROM), disk drive, solid state drive (SSD), flash memory, etc. As another example, a permanent mass storage device such as ROM, SSD, flash memory, disk drive, etc. may be a separate permanent storage device that is distinct from memory. Additionally, the memory 210 may store an operating system (OS) and at least one program code (e.g., code for the processor 110 to perform operations to be described later with reference to FIGS. 3 to 8).

These software components may be loaded from a computer-readable recording medium separate from the memory 120. Such a separate computer-readable recording medium may be a recording medium that is directly connectable to the user terminal 100, and may include, for example, a computer-readable recording medium such as a floppy drive, a disk, a tape, a DVD/CD-ROM drive, a memory card, etc. Alternatively, software components may be loaded into the memory 120 through a communication module (not shown) rather than a computer-readable recording medium. For example, the at least one program may be loaded into memory 120 based on a computer program (e.g., a computer program for the processor 110 to perform the operations to be described later with reference to FIGS. 3 to 11) installed by files provided by developers or a file distribution system that distributes installation files of applications via a communication module (not shown).

The input/output interface (not shown) may be a means for interfacing with an input/output device (e.g., keyboard, mouse, etc.) that may be connected to or included in the user terminal 100. The input/output interface (not shown) may be configured separately from the processor 110, but is not limited to this, and the input/output interface (not shown) may be configured to be included in the processor 110.

The communication module (not shown) may provide a configuration or function for the server 20 and the user terminal 100 to communicate with each other through a network. Additionally, the communication module (not shown) may provide a configuration or function for the user terminal 100 to communicate with other external devices. For example, control signals, commands, data, etc. provided under the control of the processor 110 may be transmitted to the server 20 and/or an external device through a communication module (not shown) and a network.

Meanwhile, although not illustrated in FIG. 2, the user terminal 100 may further include a display device. Alternatively, the user terminal 100 may be connected to an independent display device through wired or wireless communication to transmit and receive data between them.

FIG. 3 is a flowchart illustrating an example of a method of predicting protein-ligand binding free energy according to an embodiment.

Referring to FIG. 3, the method of predicting the protein-ligand binding free energy includes steps processed in time series in the user terminal 10, 100 or the processor 110 illustrated in FIGS. 1 and 2. Therefore, even if omitted below, the content described above regarding the user terminal 10, 100 or the processor 110 illustrated in FIGS. 1 and 2 may also apply to the method of predicting the protein-ligand binding free energy as illustrated in FIG. 3.

In step 310, the processor 110 generates the highest-probability pose for a selected protein-ligand set using a quantum mechanics-based Mining Minima algorithm.

Meanwhile, the Mining Minima algorithm may be, but is not limited to, generating the highest-probability pose using a live set including atoms of 3 Å or below in the ligand and a real set including atoms of above 3 Å and 6 Å or below in the ligand.

According to an embodiment of the present disclosure, a Mining Minima approach via the Verachem Mining Minima engine may be used to integrate quantum and molecular mechanics calculations to replace the quantum-mechanically recalculated atomic charges in the proposed pose, thereby predicting binding free energy.

According to an embodiment, the “Verachem Mining Minima (VM2) algorithm” is an “end-point” FE method, which is recently developed by Gilson et al (Chen, W., et al., Journal of chemicals theoretical computation, 2010. 6(11): p. 3540-3557). The VM2 algorithm is applied to find the best conformer by treating part of the user-defined binding pocket flexibly and holding the remaining part of the pocket rigidly. Muddana et al. predicted the binding affinity of cucurbit uril for SAMPL4 using the VM2 method and reported a high R-value correlation of 0.86 between the conventional FF and experimentally based binding affinity (Muddana, H S, et al., Journal of computer-aided molecular design, 2014. 28(4): p. 463-474). In this specification, the method according to an embodiment of the present invention will be referred to as a Qcharge-VM2 method, and the existing VM2 method will be referred to as the MM-VM2 method.

The VM2 method can refer to an “end-point” approach based on the second-generation Mining Minima approach. Thereby, the BFE of the receptor-ligand complex is distinguished from the standard chemical potential (μ°) of the ligand (L), receptor (R), and ligand-receptor complex (RL) at constant volume

$$Δ F ° = {μ}_{RL}^{◦} - {μ}_{R}^{◦} - {μ}_{L}^{◦} \qquad [ Equation 1 ]$$

In Eq. 1, μ° is the sum over all phase spaces, as defined in statistical mechanics.

$$μ° = RTLn ( ( \frac{8 {π}^{2}}{C °} ) {\sum }_{i}^{n} {Z}_{i} ) \qquad [ Equation 2 ]$$

$${Z}_{i} = {\int }_{i} {e}^{( - E ( r ) ) / RT )} dr \qquad [ Equation 3 ]$$

$$E ( r ) = U ( r ) + W ( r ) \qquad [ Equation 4 ]$$

In Equations 2 to 4, T is the absolute temperature, R is the gas constant, ° C. is the standard concentration, Zi is the configuration integral over the internal coordinates r in energy well i, E(r) is the energy as a function of r, U is the potential energy, and W is the solvation FE. And,

$$\frac{8 {π}^{2}}{C °}$$

means the degree of freedom of translation/rotation (Gilson, M K, et al., J Biophysical journal, 1997. 72(3): p. 1047-1069).

$$Δ F ° \approx Δ G ° \qquad [ Equation 5 ]$$

Meanwhile, integration over the entire phase space is included as the major reason for the high computational cost of binding affinity modeling of receptor-ligand systems. To overcome this problem, according to an embodiment of the present invention, the sum of integrals over the local energy minima of the system is used instead of the integration over the entire phase space described above. As an example, local energy minima may include tens to hundreds of minima. At this time, solvation is modeled using the generalized Born model (GB). The corrected FE for each energy minimum is calculated by adding the calculated Poisson-Boltzmann/surface area (PB/SA) solvation energy (Qiu, D., et al., The Journal of Physical Chemistry A, 1997. 101(16): p. 3005-3014, Luo, R., L. David, and M. K. Gilson, Journal of computational chemistry, 2002. 23(13): p. 1244-1253) and subtracting energy from GB used in the conformational search procedure (Chen, W., et al., Journal of chemical theory computation, 2010. 6(11): p. 3540-3557). The calibration results of the FE are checked to determine whether the new form of the pose is more stable than the previous form. This process is repeated until no new minimum is found, and the chemical potential converges to within 0.1 kcal/mol. Based on a symmetry-corrected root-mean-squared distance cutoff of 0.1 Å, conformations within 10 kcal/mol of the lowest energy conformation are filtered out and retained. Meanwhile, when the calculation is converged by the VM2 method, the average energy for all barriers (wells) is expressed as the sum of each barrier energy multiplied by the probability.

$$〈 {E}_{i} 〉 = {\sum }_{i}^{n} \frac{{e}^{- {μ}_{i}^{◦} / RT}}{{\sum }_{i}^{n} {e}^{- {μ}_{i}^{◦} / RT}} {E}_{i} \qquad [ Equation 6 ]$$

Meanwhile, PB (Poisson-Boltzmann) is calculated by setting the internal and external dielectric constants to 1 and 80, respectively.

Meanwhile, the processor 110 may set a clustering parameter such that there are no similar poses within a certain range in order to guarantee diversity of poses, but is not limited to this. As an example, the processor 110 may set the clustering parameter such that there are no similar poses within 1.5 Å RMSD in coordination with each other in the initially generated pose.

In step 320, the processor 110 replaces the charge value of the pose with atomic electrostatic potential charge (Q_{ESP}) using a quantum mechanical calculation method that predicts the atomic charges of protein residues and ligands existing in the quantum mechanical domain.

Meanwhile, the processor 110 may replace the charge value of the pose with Q_{ESP} using the QM/MM calculation method.

FF-based fixed electric charge is used in most docking methods for protein and ligand atoms. At this time, to avoid quality problems with the FF charge model, the charge of the ligand may be determined using the density functional theory (DFT) method.

By studying the effect of variable charge models obtained by Quantum Mechanics/Molecular Mechanics (QM/MM) methods on docking, the charges of the ligand atoms in the binding site may be corrected. In this way, the charge of the ligand atom, corrected for the polarization of the binding site environment, may be used to predict the conformation of the protein-ligand complex, which may provide accurate docking predictions for the lead optimization applications (Cho, A. E. and D. Rinaldo, Journal of Computational Chemistry, 2009. 30(16): p. 2609-2616, Cho, A. E., et al., Journal of computational chemistry, 2005. 26(1): p. 48-71, Chung, J. Y., J.-M. Hah, and A. E. Cho, Journal of Chemical Information Modeling, 2009. 49(10): p. 2382-2387, Park, K., N. K. Sung, and A. E. Cho, Bulletin of the Korean Chemical Society, 2013. 34(2): p. 545-548, Kim, M. and A. E. Cho, Physical Chemistry Chemical Physics, 2016. 18(40): p. 28281-28289, Cho, A. E., et al., The Journal of chemical physics, 2009. 131(13): p. 134108, Cho, A. E., et al., Journal of computational chemistry, 2005. 26(9): p. 915-931).

In step 330, the processor 110 predicts the protein-ligand binding free energy using the charge value-replaced pose.

According to an embodiment of the present disclosure, the processor may generate the highest-probability pose for a selected protein-ligand set using a quantum mechanics-based Mining Minima algorithm, replace a charge value of the pose with atomic electrostatic potential charge (Q_{ESP}) using a quantum mechanical calculation method predicting the atomic charges of protein residues and ligands existing in a quantum mechanical domain, and predict the protein-ligand binding free energy.

As an example, the processor may generate a highest-probability pose using the MM-VM2 method and predict the protein-ligand binding free energy using the Qcharge-VM2 method.

Hereinbelow, preferred embodiments are provided to aid understanding of the present disclosure. However, the following embodiments are provided only for easier understanding of the present disclosure, and the content of the present disclosure is not limited by the following embodiments.

### EXPERIMENTAL EXAMPLE 1

In Experimental Example 1, an experiment was performed to compare an embodiment of the present disclosure with a study by Wang et al. (Wang, L., et al., Journal of the American Chemical Society, 2015. 137(7): p. 2695-2703.), a study by Gapsys et al. (Gapsys, V., et al., Chemical Science, 2020. 11(4): p. 1140-1152), and a study by Li et al. (Li, Z., et al., Journal of medicinal chemistry, 2019. 62(4): p. 2099-2111).

To evaluate the method of predicting binding free energy according to an embodiment of the present disclosure, a series of known co-crystal structures of HIV-1protease, CDK2, JNK1, BACE1, Thrombin, P38 and TYK2, including 147 different ligands, were used. All samples, except HIV-1, were downloaded from GitHub (https://github.com/openforcefield/protein-ligand-benchmark). The common scaffold of the ligands used in the calculations of the VM2 method was obtained from Supporting Information, Figure S1. All experimental Kᵢ values were obtained from IC50 using the Cheng-Prusoff equation (Yung-Chi, C. and W. H. Prusoff, J Biochemical pharmacology, 1973. 22(23): p. 3099-3108, Frush, E. H., S. Sekharan, and S. Keinan, The Journal of Physical Chemistry B, 2017. 121(34): p. 8142-8148).

In Experimental Example 1, the ΔGoffset value in Equation 7 was used to compare the results according to an embodiment of the present invention with the results according to other methods. In Experimental Example 1, the ΔGoffset value in Equation 7 was used to compare results according to an embodiment of the present disclosure with results according to other methods.

$$Δ {G}_{offset , i}^{calc} = Δ {G}_{i}^{calc} - \frac{1}{N} {\sum }_{i = 1}^{N} ( Δ {G}_{i}^{calc} - Δ {G}_{i}^{exp} ) \qquad [ Equation 7 ]$$

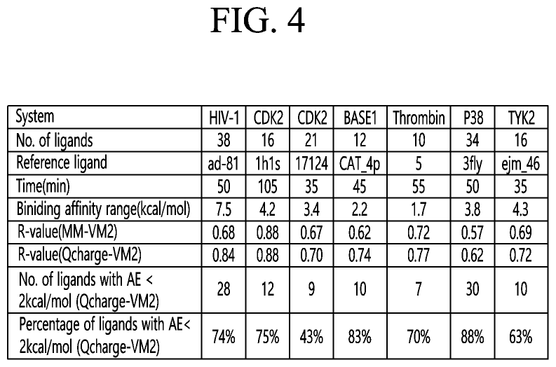

In Equation. 7, ‘N’ means the number of ligands, ‘calc’ means the calculated value, and ‘exp’ means the experimental value. In addition, the subscript ‘i’ refers to the value for the i-^{th} ligand. Here, ΔG_{offset}^{calc} Values were generated by adjusting individual ΔG values by the calculated ΔG (mean signed error, MSE). FIG. 4 is a diagram illustrating the relative binding free energy predicted by a processor according to an embodiment.

Referring to FIG. 4, the data set used in the experiment and the Pearson correlation between the calculated BFE and the observed IC50 or Kᵢ value may be confirmed.

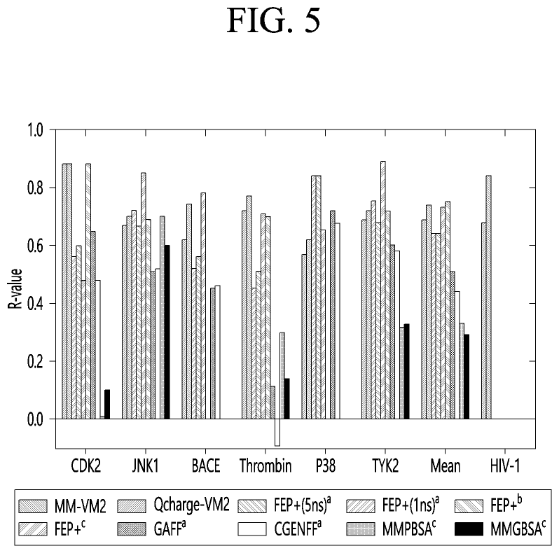

FIG. 5 is a diagram for comparing relative binding free energy predicted by a processor according to an embodiment.

Referring to FIG. 5, a scatter plot of the predicted BFEs and experimental BFEs for the entire data set, compared to methods such as FEP+, General Amber Force Field (GAFF), CHARMM General Force Field (CGenFF), MM-PB-SA, and MM-GB-SA, can be seen (Wang, L., et al., Journal of the American Chemical Society, 2015. 137(7): p. 2695-2703, Gapsys, V., et al., Chemical Science, 2020. 11(4): p. 1140-1152, Li, Z., et al., Journal of medicinal chemistry, 2019. 62(4): p. 2099-2111).

HIV-1 protease is a promising target for antiviral treatment in AIDS patients. A total of 38 ligands with Kᵢ values ranging from 0.0008 nM to 238 nM were used. Referring to FIG. 5, it can be seen that the BFE calculated using the Qcharge-VM2 method has an R-value of 0.84, which correlates very well with the tested BFE. It can be seen that the protein-ligand system for the HIV-1 protease significantly improves both the R-value and the predicted ΔG value of below 2 kcal/mol. According to the predictions of the Qcharge-VM2 method, 74% of the ligands have an absolute error (AE) of below 2 kcal/mol, which is much better than the MM-VM2 method (63%) (Ali, A., et al., Journal of medicinal chemistry, 2006. 49(25): p. 7342-7356.).

Cyclin-dependent kinases-2 (CDK2) is a member of the protein kinase family and play an essential role in regulating the division of eukaryotic cells (Hardcastle, I. R., et al., Journal of medicinal chemistry, 2004. 47(15): p. 3710-3722, Chohan, T. A., et al., Current medicinal chemistry, 2015. 22(2): p. 237-263). Experiments were performed on a set of 16 ligands with Kᵢ values ranging from 0.0054 μM to 6.8 μM. Applying the Qcharge-VM2 method to the CDK2 ligand system improved the number of predicted energies with an AE of below 1 kcal/mol from 31% to 44%. On the other hand, the MM-GB/PB-SA method showed very poor results.

JNK-1 (c-Jun N-terminal kinases) have been implicated in the pathology of various diseases such as stroke, asthma, type 2 diabetes, and Alzheimer's disease. In Experimental Example 1, 21 ligands with Kᵢ values ranging from 14 nM to 4400 nM were used (Cumming, J. N., et al., Bioorganic medicinal chemistry letters, 2012. 22(7): p. 2444-2449). Referring to FIG. 5, the R-value predicted by the Qcharge-VM2 method is 0.70, which is better than the MM-VM2 prediction, and in the Qcharge-VM2 calculation, 76% of the ligands have an AE below 2.5 kcal/mol, while in the MM-VM2 calculation, only 52% of the ligands have the same AE range.

BACE1 (β-APP cleaving enzyme 1) acts as a potential disease modifier in Alzheimer's disease. In Experimental Example 1, 12 ligands with Kᵢ values ranging from 0.09 μM to 3.8 μM were used. Referring to FIG. 5, it can be seen that the correlation calculated by the Qcharge-VM2 method is 0.12 higher than the MM-VM2 method.

Thrombin plays an important role in the pathogenesis of arterial thrombosis (Lee, C. J. and J. E. Ansell, British journal of clinical pharmacology, 2011. 72(4): p. 581-592, Baum, B., et al., Journal of molecular biology, 2009. 390(1): p. 56-69). Experimental Example 1 was performed using ligands with Ki values of 0.21 μM, 3.55 μM, and 10 μM. Referring to FIG. 5, it can be seen that better correlation was obtained when performing the Qcharge-VM2 method than the other methods. It can also be seen that 60% of the ligands exhibit an AE of below 1 kcal/mol by the Qcharge-VM2 method, compared to only 40% by the MM-VM2 method.

The P38 mitogen-activated protein (MAP) kinase plays an important role in regulating cytokine production (Schett, G., J. Zwerina, and G. Firestein, Annals of the rheumatic diseases, 2008. 67(7): p. 909-916.). In Experimental Example 1, 34 ligands with Kᵢ values ranging from 1 nM to 594 nM were used. It can be seen that the protocol correlation for P38 by the Qcharge-VM2 method is 0.05 higher than that of MM-VM2. The energy predicted by Qcharge-VM2 calculations shows that 21% of the ligands have an AE greater than 1.5 kcal/mol, compared to 44% for MM-VM2.

TYK2 (Nonreceptor tyrosine-protein kinase 2) is involved in both inflammation and immunity because cytokine receptors may regulate an immune cell function. In Experimental Example 1, 12 ligands with Kᵢ values ranging from 0.0048 μM to 3.5 μM and 4 ligands with Kᵢ values ranging from 2.5 nM to 9.1 nM were used. In calculations using the Qcharge-VM2 method, below 30% showed an AE greater than 2 kcal/mol, whereas in calculations using the MM-VM2 method, approximately 50% showed an AE greater than 2 kcal/mol.

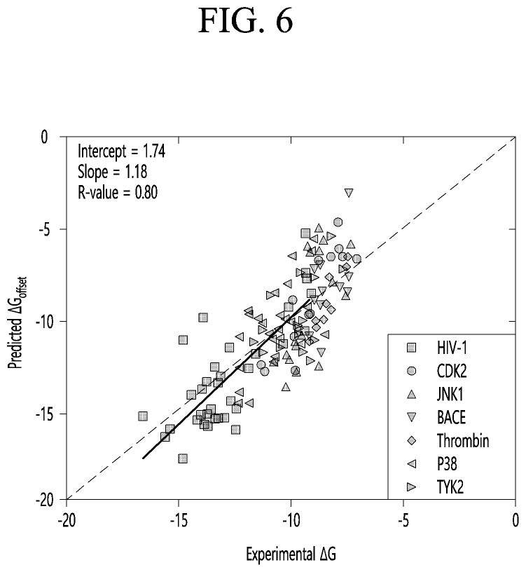

FIG. 6 is a diagram illustrating the experimental binding energy and the binding energy predicted by the MM-VM2 method.

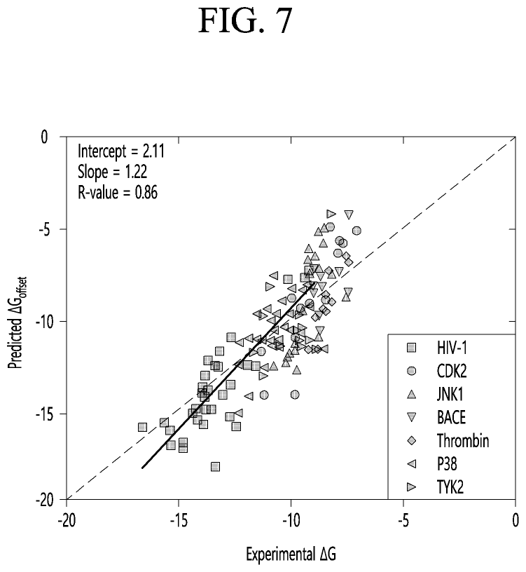

FIG. 7 is a diagram illustrating the experimental binding energy and the binding energy predicted by a processor according to an embodiment using the Qcharge-VM2 method.

Referring to FIGS. 6 and 7, it can be seen that the best-fit lines (red lines) for the 140 ligands (excluding the seven outliers) by the seven methods exhibit slopes of 1.18 and 1.22 and intercepts of 1.74 and 2.11 for the MM-VM2 method and Qcharge-VM2 method, respectively. The R-values for both methods were 0.80 and 0.86 for 140 different ligands. As such, it can be seen that the prediction of binding free energy is improved by the Qcharge-VM2 method.

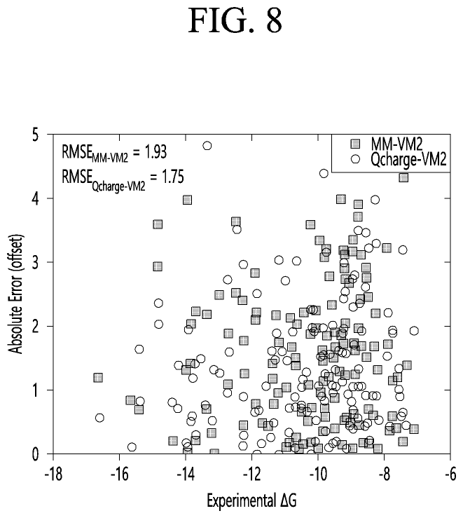

FIG. 8 is a diagram illustrating experimental binding energy and an absolute error offset according to an embodiment.

Referring to FIG. 8, it can be seen that about 93% of the 147 measured energies differ by below 4 kcal/mol from the experimental measurements. Additionally, the root mean square error (RMSE) of the two methods corresponds to 1.93 and 1.75 kcal/mol. As such, it can be seen that the prediction of binding free energy is improved by the Qcharge-VM2 method.

According to an embodiment of the present disclosure, it can be seen that the R-value, the RMSE, etc. may be improved by replacing charges by the Qcharge-VM2 method. Additionally, according to an embodiment of the present disclosure, it can be seen that the Qcharge-VM2 method has the same or better accuracy compared to the expensive FEP+ method.

Li et al. reported that ABFE calculations took an average of 10 hours per ligand using eight Nvidia Geforce GTX-580 GPUs for practical structure-based drug design (Li, Z., et al., Journal of medicinal chemistry, 2019. 62(4): p. 2099-2111). In addition, Wang et al. reported that 8 Nvidia GTX-780 GPUs were needed for 6 hours for each perturbation (Wang, L., et al., Journal of the American Chemical Society, 2015. 137(7): p. 2695-2703). Additionally, other methods required 20 hours on a single CPU system for a single conformer to calculate the binding energy through coupled MD simulations and QM/MM calculations (Frush, E. H., S. Sekharan, and S. Keinan, The Journal of Physical Chemistry B, 2017. 121(34): p. 8142-8148). In addition, the completion of the computation for one ligand with another FEP+ protocol proposed by Steinbrecher required 100 minutes of execution on 30 GPUs (Steinbrecher, T. B., et al., Journal of chemical information modeling, 2015. 55(11): p. 2411-2420). On the other hand, according to an embodiment of the present disclosure, the FE calculation took an average of about 50 minutes per ligand on an Intel® Xeon® CPU E5-2680 v3, 2.50 GHz with 12 MPI processes, as illustrated in FIG. 4.

According to an embodiment of the present disclosure, it can be seen that in predicting protein-ligand binding free energy, reasonable correlation is generated with less time consumption.

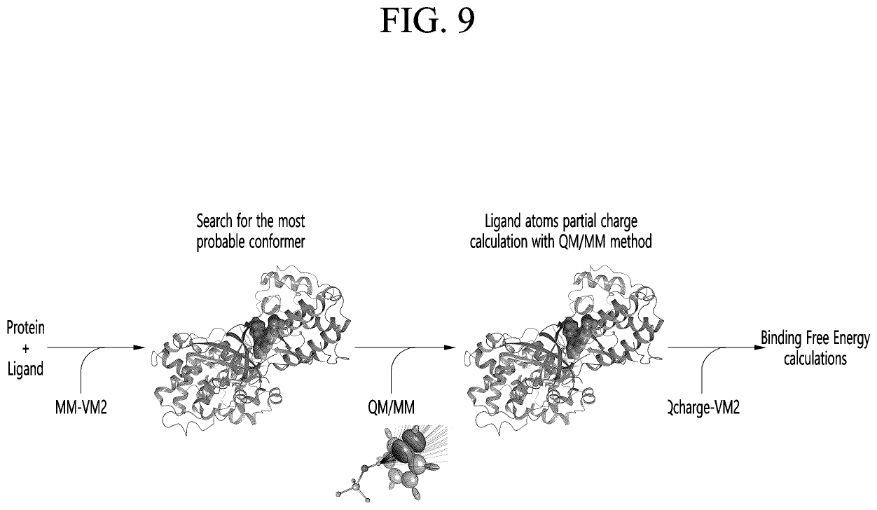

FIG. 9 is a diagram illustrating another example of a method of predicting protein-ligand binding free energy according to an embodiment.

Referring to FIG. 9, the processor 110 generates the highest-probability pose for a selected protein-ligand set using a quantum mechanics-based Mining Minima algorithm. For example, the processor 110 may search for the most probable conformer using a MM-VM2 method. Then, the processor 110 replaces the charge value of the pose with atomic electrostatic potential charge (Q_{ESP}) using a quantum mechanical calculation method that predicts the atomic charges of protein residues and ligands existing in the quantum mechanical domain. For example, the processor 110 may calculate a ligand atoms partial charge using a QM/MM calculation method. Then, the processor 110 predicts the protein-ligand binding free energy using the charge value-replaced pose.

As described before, the processor 110 predicts the protein-ligand binding free energy. The user 30 can confirm the highly accurate and precise protein-ligand binding free energy with less time consumption at low cost.

Meanwhile, the above-described method can be written as a program that can be executed on a computer, and can be implemented in a general-purpose digital computer that operates the program using a computer-readable recording medium. Additionally, the data structure used in the above-described method can be recorded on a computer-readable recording medium through various means. The computer-readable recording media includes storage media such as magnetic storage media (e.g., ROM, RAM, USB, floppy disk, hard disk, etc.) and optical read media (e.g., CD-ROM, DVD, etc.).

Those skilled in the art related to the present disclosure will understand that the above-described embodiments can be implemented in modified forms without departing from the essential technical scope of the present disclosure. Therefore, the disclosed methods should be considered from an explanatory rather than a limiting perspective, and the scope of right is indicated in the claims, not the foregoing description, and should be interpreted to include all differences within the equivalents of the claims.

According to the problem-solving means of the present disclosure described above, the present disclosure generates the highest-probability pose for a selected protein-ligand set using a quantum mechanics-based Mining Minima algorithm, replaces a charge value of the pose with atomic electrostatic potential charge (Q_{ESP}) using a quantum mechanical calculation method predicting the atomic charges of protein residues and ligands existing in a quantum mechanical domain, and predicts the protein-ligand binding free energy using the charge value-replaced pose, so it is possible to predict the highly accurate and precise protein-ligand binding free energy with less time consumption at low cost.

The effects of the present disclosure are not limited to those mentioned above, and other effects not mentioned will be clearly understood by those skilled in the art from the above description.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A method of predicting protein-ligand binding free energy, the method comprising:
generating a highest-probability pose for a selected protein-ligand set using a quantum mechanics-based Mining Minima algorithm;
replacing a charge value of the highest-probability pose with atomic electrostatic potential charge (Q_{ESP}) using a quantum mechanical calculation method predicting atomic charges of protein residues and ligands existing in a quantum mechanical domain; and
predicting protein-ligand binding free energy using the highest-probability pose whose charge value has been replaced with the atomic electrostatic potential charge.

2. The method of claim 1, wherein
the Mining Minima algorithm generates the highest-probability pose using a live set including atoms of 3 Å or below in the ligand and a real set including atoms of above 3 Å and 6 Å or below in the ligand.

3. The method of claim 1, wherein
the quantum mechanical domain includes protein residues and ligands at a binding site between a protein and a ligand.

4. The method of claim 1, wherein
the replacing comprises replacing the charge value of the highest-probability pose with the atomic electrostatic potential charge (Q_{ESP}) using a QM/MM calculation method.

5. A computer-readable recording medium recording a program for executing the method of claim 1 on a computer.

6. A computing apparatus comprising:
at least one memory; and
at least one processor,
wherein the at least one processor is configured to:
generate a highest-probability pose for a selected protein-ligand set using a quantum mechanics-based Mining Minima algorithm;
replace a charge value of the highest-probability pose with atomic electrostatic potential charge (Q_{ESP}) using a quantum mechanical calculation method predicting the atomic charges of protein residues and ligands existing in a quantum mechanical domain; and
predict protein-ligand binding free energy using the highest-probability pose whose charge value has been replaced with the atomic electrostatic potential charge.

7. The computing apparatus of claim 6, wherein
the Mining Minima algorithm generates the highest-probability pose using a live set including atoms of 3 Å or below in the ligand and a real set including atoms of above 3 Å and 6 Å or below in the ligand.

8. The computing apparatus of claim 6, wherein
the quantum mechanical domain includes protein residues and ligands at a binding site between a protein and a ligand.

9. The computing apparatus of claim 6, wherein
the quantum mechanical calculation method comprises a QM/MM calculation method.
